(19) United States
(12) Patent Application Publication
(10) Pub. No.: US 2013/0037021 A1
(43) Pub. Date: Feb. 14, 2013

(54) INHALER

(71) Applicants: Richard D. Brewer, Loughborough (GB); Craig Davidson, Cumbria (GB); Graham R. Purkins, West Sussex (GB); Stephen J. Howgill, Thurcaston (GB)

(72) Inventors: Richard D. Brewer, Loughborough (GB); Craig Davidson, Cumbria (GB); Graham R. Purkins, West Sussex (GB); Stephen J. Howgill, Thurcaston (GB)

(73) Assignee: 3M INNOVATIVE PROPERTIES COMPANY, ST. PAUL, MN (US)

(21) Appl. No.: 13/642,737

(22) PCT Filed: Apr. 21, 2011
(86) PCT No.: PCT/US11/33379

§ 371 Date: Oct. 22, 2012

(30) Foreign Application Priority Data
Apr. 23, 2010 (GB) 1006759.3

(51) Int. Cl. A61M 15/00; A61M 11/04; A61M 16/14; A61M 15/08

(52) U.S. Cl. 128/200.23; 128/203.12; 128/203.15

(57) ABSTRACT

An actuator (2) for an inhaler (1) comprising a longitudinally extending body (3), a delivery passage at (or near to) a first end of the body (3) and a retained cover (7) for the delivery passage, the actuator (2) having at least one profile (9) arranged to guide the cover (7) from a closed position occluding at least a user-opening (6) of the delivery passage to a remote location in which the user-opening (6) of the delivery passage is unoccluded wherein the cover (7) and the at least one profile (9) are configured and arranged such that, in use of the actuator (2), in the guided movement of the cover (7) from the closed position to the remote location, the cover (7) is glided by the user towards the second end of the body (3).

### FIELD

This description relates to an actuator used in a dispensing device as well as a dispensing device arranged to deliver a medicament to the respiratory system of a patient in response to an inhalation of the patient or by dispensing intranasally. Such dispensing devices are commonly known as inhalers, and include pressurized metered dose inhalers, dry powder inhalers and portable nebulizers. Such devices typically include a container containing medicament for delivery to the patient, and such containers are typically, in part or completely housed and/or supported within an actuator that typically includes a delivery passage generally adapted for oral or intranasal delivery.

### BACKGROUND

Pressurized metered dose inhalers (pMDI) are well known and may be used for delivering medication in the form of aerosols to the patient, where the delivery of the medicament may be oral or nasal. Such an inhaler commonly comprises a canister containing the medicament aerosol formulation, an actuator and a delivery passage. The canister contains the aerosol formulation, either as a solution or suspension, in the form of one or more drugs and propellant, and optionally excipients, selected from co-solvents, surfactants, stabilizing substances (for chemical or physical stability) and flavourings. The canister also comprises a metering valve arranged to deliver a metered dose of the medicament on actuation of the inhaler. The actuator typically comprises a housing, generally made of a plastic material, within which the canister is located. In oral inhalers the actuator has a delivery passage in the form of a mouthpiece which is placed in the patient's mouth and through which the medicament passes on being dispensed. The patient places the mouthpiece in their mouth and breathes in creating an air flow from the actuator through the mouthpiece and into the mouth and lungs. At the same time the patient actuates dispensation of the medicament from the canister. Actuation may occur as a result of the inhalation or the patient may depress the canister towards the actuator. Nasal actuators operate in a similar fashion where instead of a mouth piece the actuator is provided with a nose piece for delivery of the medicament to the nasal passages.

It is known to provide removable covers for the mouth/nose piece and to remove the cover so that the inhaler can be used. If the cover is separable from the actuator the cover may be lost. If the delivery passage is left uncovered dirt may enter the inhaler and may be inhaled by the patient or interfere with the dispensing of drug during a subsequent use of the inhaler.

WO2005046774 discloses an inhaler comprising a body (actuator) which includes a mouthpiece and a cap which can be placed in a position to substantially occlude the mouthpiece. The cap is attached to the body by a strap which underlies the base of the body and can pivot relative to the body. The cap is arranged to slide on the strap such that the cap must translate away from the mouthpiece prior to pivoting the strap to move the cap so that the mouthpiece is accessible.

U.S. Pat. No. 3,429,310 discloses an aerosol inhaler including a base (actuator), a swivelly mounted cap on the base, a lateral open-ended inhalating tube on the base, wherein the cap is provided with a lateral projection to overlie or selectively be free of the inhalating tube when the cap is relatively swiveled to desired position relative to the base, and wherein the aerosol product is discharged through the inhalating tube when the base and cap are moved slightly towards each other to relatively depress the aerosol stem.

### SUMMARY

It has been found that caps of types described above are often clumsy and inconvenient to use, and often obstruct or hinder actuation of the inhaler by a patient (e.g. by blocking the user's hand). In some cases this can be particularly critical for asthma patients in a stress situation quickly needing to take a dose of medicament, and in other cases patients may simply stop using the cap leaving the mouth- or nasal-piece uncovered between actuations thus defeating the original purpose of the cap.

According to a first aspect of the description, there is provided an actuator for an inhaler comprising a longitudinally extending body, a delivery passage at (or near to) a first end of the body and a retained cover for the delivery passage, the actuator having at least one profile arranged to guide the cover from a closed position occluding at least a user-opening of the delivery passage to a remote location in which the user-opening of the delivery passage is un-occluded wherein the cover and the at least one profile are configured and arranged such that, in use of the actuator, in the guided movement of the cover from the closed position to the remote location, the cover is glided by the user towards the second end of the body.

Actuators in accordance with the description are advantageous in that their use with inhalers allows for an intuitive mode of operation in that the user can move the cover out of the way of the delivery passage in a single movement by pushing it or lifting it up in the general direction of the second end of the actuator body. For example the provision of at least one profile in actuators guides the cover from a closed position to a remote location (i.e. an opened position) facilitates ease of operation. Moreover, the guided gliding movement of the cover by the user will naturally cause the cover to move to its correct opened location (advantageously on the body of actuator itself) without the user needing to be given specific instructions as to the location the cover should be moved to before administering medicament. Further while the cover is retained (i.e. securely retained on the body) and thus can not be lost, the gliding movement of the cover to the remote location allows flexibility in the design of the cover.

In addition, such actuators allow for a convenient and easy one-handed operation, allowing the user to operate the inhaler without changing their grip on the device. For example in use in pressurized metered dose inhalers by using the thumb to push/lift the cover towards the second end of the actuator body, the user can in a continued movement of their thumb then position their thumb at the base of the inhaler canister (said base typically extending from the second end of the actuator body) to actuate the inhaler.

Favourably the cover substantially encompasses the delivery passage in its closed position, In other words, the cover is favourably arranged and configured such that in its closed position it covers the user-opening of the delivery passage and substantially surround the delivery passage on at least three sides of the delivery passage, thus providing advantageous extensive protection of the delivery passage while the inhaler is not in use by the patient. An appropriately positioned and sized aperture may favourably be incorporated in such a cover to facilitate the cover-opening. Desirably such an aperture is a close fit to the delivery passage in the cover's closed position (e.g. the aperture may be sized to allow the delivery passage to just pass through).

In a further aspect of the description there is provided an actuator for an inhaler comprising a longitudinally extending body, a delivery passage at (or near to) a first end of the body and a retained cover for the delivery passage, the actuator having at least one profile arranged to guide the cover from a closed position occluding the delivery passage to a remote location on the body, wherein in its closed position the cover substantially encompasses the delivery passage.

Favourably actuators described herein may be further provided with a retainer for additionally holding the cover in its closed position (“closed retainer”). Favourably actuators may be further provided with a retainer for additionally holding the cover in its remote location (“open retainer”). In preferred embodiments, actuators comprise both an additional closed retainer and open retainer, so that the cover may be additionally retained in either its closed position or its remote location as appropriate, reducing any inadvertent or unintended movement of the cover. In other words movement is thus favourably restricted until the patient wishes to move the cover, and in this way, the cover is held in a particular position until the patient makes a positive decision to open and/or close the cover.

In preferred embodiments of actuators described herein, the cover and body may be provided with co-operating formations to retain the cover in the closed position and/or the remote location. The co-operating formations may be incorporated in the at least one profile or may be separate therefrom. One or both co-operating formations may be resilient so as to engage/disengage automatically in response to movement of the cover towards and away from the relevant position. For example, the co-operating formation on the cover may be located on a flexible tab to cooperate resiliently with the co-operating formation on the body such that the formations engage/disengage in response to a positive action from the patient to move the cover. The co-operating formations may comprise a projection and a recess or hole although any suitable formations may be employed.

Preferably, the cover of actuators described herein extends around the body, said body extending longitudinally and defining a longitudinal axis. Desirably the cover is shorter than the body along the body's longitudinal axis. Desirably the cover generally has a form of a closed ring or opened ring, i.e. topologically equivalent to a closed or opened ring (a sector of a ring). Such a configuration enhances stability and/or reduces the chances of unwanted detachment of the cover from the body. The body and/or the cover may have a cross section, orthogonal to the longitudinal axis of the body, which is circular or oval.

In some embodiments, the cover is in the form of a closed ring that encircles the body. In other words, the ring may thus completely surround the body. In alternative embodiments, the cover may be generally in the form of a sector of a ring, thus not completely encircling the body. In such embodiments the cover may thus partially surround the body, preferably so that the angle subtended by the sector of the ring at the longitudinal axis of the body is greater than 180 degrees, more preferably greater than 240 degrees, even more preferably greater than 290 degrees, or most preferably greater than 325 degrees. (It will be appreciated that the angle subtended by a closed ring at the longitudinal axis of the body is 360 degrees.)

The at least one profile of actuators described herein may be favourably provided on an outer surface of the body. The at least one profile may favourably be in the form of a track arranged to guide the cover to and from the closed position and the remote location. Appropriately the cover may favourably have at least one formation on an inner surface that is co-operable with the track on the outer surface of the body as the cover moves between the closed position and the remote location. For example, the track may comprise a groove in the outer surface of the body and the formation may comprise a projection on the inner surface of the cover that is received in and slidable along the groove to guide the cover between the closed position and the remote location. Alternatively the at least one profile may comprise a contour change on the outer surface of the body. A further alternative is that the profile may comprise a ridge or other protuberance on the outer surface of the body. In some embodiments the at least one profile may be provided on the inner surface of the cover. For example the inner surface of the cover may be provided with a track and the outer surface of the body may be provided with a co-operating formation.

The at least one profile of actuators described herein may include a single directional component (e.g. the at least one profile may run just longitudinally). Alternatively the at least one profile may include two or more directional components (e.g. the at least one profile may run, in order from the closed position to the remote position of the cover, first longitudinal then helical or first longitudinal then rotational or first in one lateral direction then in a second lateral direction). Desirably the at least one profile comprises a longitudinal component relative to the actuator body and/or a lateral component relative to the actuator body and/or a helical component relative to the actuator body and/or a rotational component relative to the actuator body.

Here and in the following “lateral” is understood to be a direction that is inclined at an angle of no more than 60° (preferably no more than 45°, more preferably no more than 35°) relative to the longitudinal axis of the body of the actuator.

Here and in the following “rotational” is understood to be a direction of rotation that is perpendicular to the longitudinal axis of the body of the actuator.

In preferred embodiments of actuators described herein, the actuator has at least one pair of profiles. In such embodiments the at least one pair of profiles are favourably provided on an outer surface of the body. Desirably the at least one pair of profiles are provided on opposing sides of the body. Such at least one pair of profiles are desirably arranged symmetrically with respect to the delivery passage. Favourably the at least one pair or profiles are opposed such that the said at least one pair is bisected by a vertical plane of symmetry bisecting the delivery passage. Arranging the profiles in one or more pairs on opposing sides of the body provides greater stability for the movement of the cover between the closed position and the remote location. In some embodiments, two or more pairs of profiles may be provided on opposing sides of the body. For such embodiments pairs of profiles are preferably similar and are preferably spaced apart.

In actuators described herein the guided movement of the cover from the closed position to the remote location may desirably include a longitudinal component relative to the actuator body and/or a lateral component relative to the actuator body and/or a helical component relative to the actuator body and/or a rotational component relative to the actuator body.

Desirably the guided movement of the cover from the closed position to the remote location does not include a rotational component relative to the actuator body as a first component of said movement.

Desirably the first motion of the cover in its guided movement from the closed position to the remote location is longitudinal or lateral or helical relative to the actuator body.

A first motion vertically (be it longitudinally, laterally or helically) towards the second end of the body further facilitates intuitive use and/or ease in opening (in particular for example one-handed with a thumb).

A number of directions of movement are possible to achieve the desired aim of moving the cover so that the user opening is accessible when the inhaler is to be used. For example in actuators described herein the motion of the cover in its guided movement from the closed position to the remote location may favourably include one of the following motions:
longitudinal relative to the actuator body;
longitudinal followed by lateral, helical and/or rotational relative to the actuator body;
lateral relative to the actuator body;
lateral followed by longitudinal, helical, rotational and/or a second lateral motion relative to the actuator body;
helical relative to the actuator body; and
helical following by longitudinal, lateral, rotational and/or a second helical motion relative to the actuator body.

Favourably the outer surface of the cover of actuators described herein comprises one or more user-gripping regions further facilitating easy and/or one-handed operation. Such user-gripping regions of the inhaler may be opposed and suitable for right-handed or left-handed users. They may be provided as structured features e.g. shallow ribs, or matte finish, or may be achieved using high surface energy coating.

Favourably the actuator body of actuators described herein is adapted to be able to house and/or support a container containing medicament for delivery to the respiratory system of the user. Preferably the actuator is open at the second end of the body distal from the delivery passage. Desirably the actuators described herein are advantageously adapted and configured to allow a canister (e.g. a pMDI-canister) to be reversibly inserted (i.e. inserted, removed, re-inserted, etc.) into the second end of actuator without any hindrance by the cover. For favoured embodiments where actuators are used in pMDIs, desirably such actuators are adapted for supporting and containing a pMDI-canister.

Favourably the delivery passage of actuators described herein is adapted for oral delivery or intranasal delivery. The delivery passage preferably projects from the body such that the user opening is accessible to the patient when the cover is moved to the remote location. In particular, the delivery passage of the actuator is desirably in the form of a mouth piece for oral delivery or a nose piece for intranasal delivery of a medicament (such forms are generally used in metered dose inhalers, in particular pMDIs).

An additional aspect of the present description includes a metered dose inhaler comprising an actuator described herein. While actuators described herein are favourably suited for use in metered dose inhalers such as pMDIs, dry powder inhalers and nebulizers, they are particularly suited for use in pMDIs.

### BRIEF DESCRIPTION OF THE DRAWINGS

The description will now be furthered by way of example only with reference to the accompanying drawings in which like reference numerals are used to indicate corresponding parts and wherein:

FIG. 1a is a side view of an inhaler in accordance with a first exemplary embodiment in accordance with the present the description showing the cover in a closed position;

FIG. 1b is a side view of the inhaler of FIG. 1a with the cover in a remote location;

FIG. 2a is a front view of an inhaler in accordance with another exemplary embodiment in accordance with the present description with the cover in a closed position;

FIG. 2b is a side view of the inhaler of FIG. 2a;

FIG. 2c is a cross section along the line A-A of FIG. 2a;

FIGS. 2d to 2e are perspective views of the inhaler of FIG. 2a;

FIGS. 2f to 2h are perspective views of the inhaler of FIG. 2a now with the cover in its remote location;

FIG. 2i is a side view of the inhaler of FIG. 2a with the cover in an intermediate position between the closed position and the remote location;

FIGS. 3a to 3c show a further exemplary embodiment in accordance with the present description with the cover in its closed position, intermediate position and its remote location, respectively;

FIG. 4a is a side view of an inhaler according to a yet another exemplary embodiment in accordance with the present description with the cover in a closed position;

FIG. 4b is a side view of the inhaler of FIG. 4a with the cover in a remote location;

FIG. 4c is a side view of the inhaler of FIG. 4a with the cover removed.

### DETAILED DESCRIPTION OF THE PREFERRED EMBODIMENTS

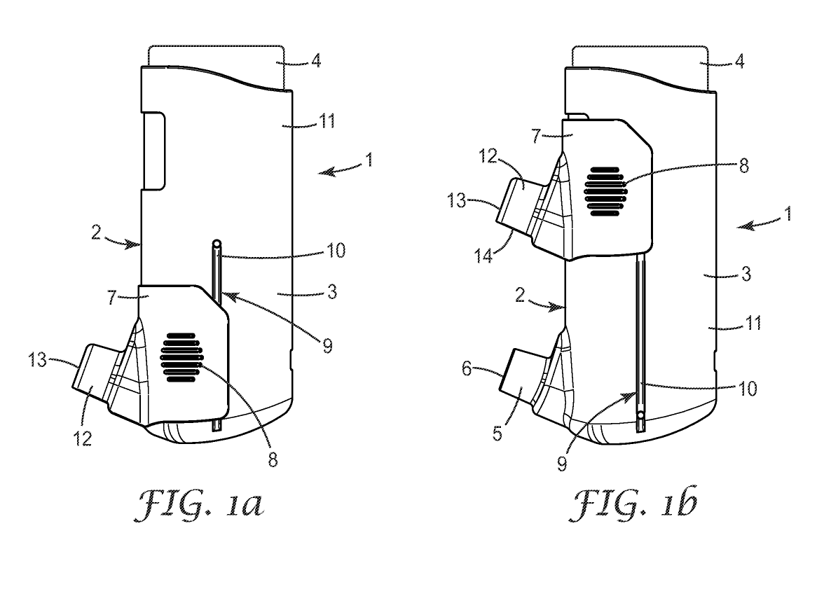

An inhaler 1 in accordance with a first exemplary embodiment in accordance with the present description is shown in FIGS. 1a and 1b.

The inhaler comprises an actuator 2 and a canister 4 located within the actuator. The actuator includes a body 3. The actuator 2 has a delivery passage in the form of a nose piece 5 located and provided as an extension at the first end of the body 3. The canister 4 can be reversibly inserted into the actuator 2 through an opening at the second end of the body 3. The body together with the delivery passage may be made of plastics, for example a plastic moulding.

A delivery channel extends within the nose piece and terminates in a user opening or discharge orifice 6 for intranasal delivery of a medicament. It will be understood that the actuator could have a body with a mouth piece for oral delivery of a medicament in place of the nose piece.

The actuator 2 further comprises a cover 7 for the nose piece 6. The cover 7 is shown in a closed position in FIG. 1a and at its remote location on the body 3 of the actuator 2 in FIG. 1b. The remote location may be referred to as the open or opened position.

The cover 7 is in the form of an open ring such that the sector of the ring subtends an angle of least 180 degrees (here approximately 215 degrees) at the longitudinal axis of the body and partially encircles the body 3 of the actuator. The cover 7 is provided with finger grips 8 on either side of the cover. The cover may be made of plastics, for example a plastic moulding.

The actuator 2 is provided with a pair of opposed profiles 9 (one only visible) in the form of tracks 10 on opposing sides of the body 3. Each track 10 comprises a groove that runs longitudinally along an outer surface 11 of the body 3. The grooves are preferably arranged symmetrically with respect to a vertical plane bisecting the nose piece 5.

The cover 7 is guided as it moves from the closed position shown in FIG. 1a to the remote location shown in FIG. 1b by formations (not shown) such as lugs or projections on the inner surface of the cover 7 that are received in and slide along the grooves. The engagement of the lugs in the grooves retains the cover 7 on the body 3.

The cover 7 is releasably held in its closed position and its remote location by engagement of the lugs with retainer formations (not shown) at the ends of the grooves. The cover 7 partially encircles the body 3 and the cover 7 may flex so that the lugs can engage/disengage the retainer formations resiliently, for example with a snap action, as the cover 7 is moved towards and away from a retained position. In this way, the cover 7 is held in the retained positions at the end of travel and the user is provided with a positive indication that the cover 7 is secured.

The cover 7 has a forwardly extending projection 12 arranged to extend over the nosepiece 5 in the closed position. The projection 12 has an end 13 that extends across the discharge orifice 6 of the nosepiece 5 and occludes the discharge orifice 6 in the closed position.

The cover 7 moves from the closed position to the remote location in a longitudinal direction relative to the body 3. In the remote location the cover 7 is displaced from the nosepiece 5 which can be used by a patient for the delivery of medication from the canister 4 without the cover 7 interfering or obstructing use of the inhaler. The cover 7 is returned to the closed position by a reverse of the above operation.

In this embodiment the grooves extend substantially parallel to the longitudinal axis and the cover 7 is guided lengthwise of the body 3 so that the projection 12 remains axially aligned with and spaced from the nosepiece 5 in the remote location.

The cover 7 substantially encompasses the nose piece 5 in the closed position. The discharge orifice 6 is occluded by the projection 12 of the cover 7 preventing the ingress of dirt or other foreign bodies into the delivery passage. In addition the projection 12 extends over an upper portion of the nosepiece 5 and over each side of the nosepiece. An aperture 14 (not visible) is provided in a lower portion of the cover 7 for passage of the nose piece 5 as the cover moves to and from the closed position of FIG. 1a.

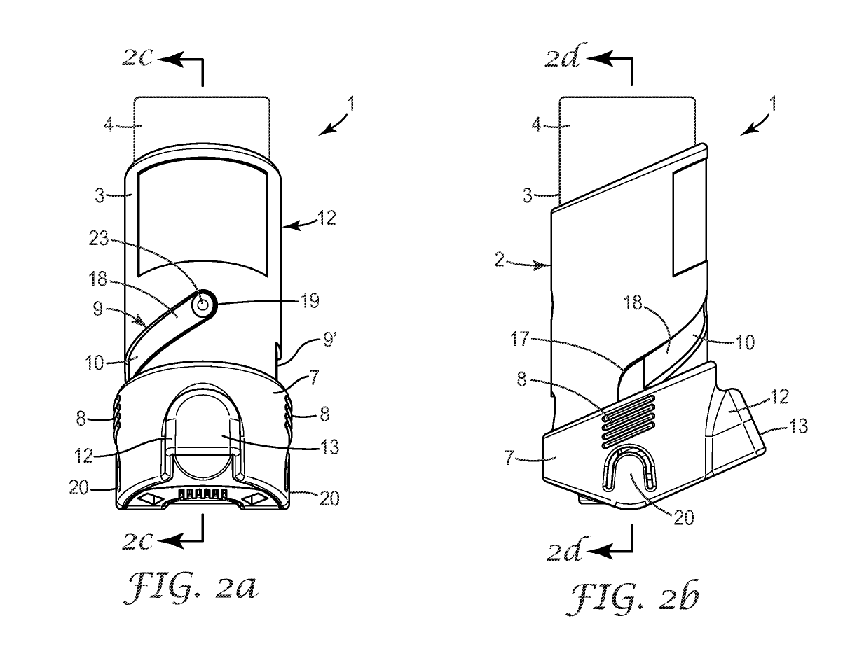

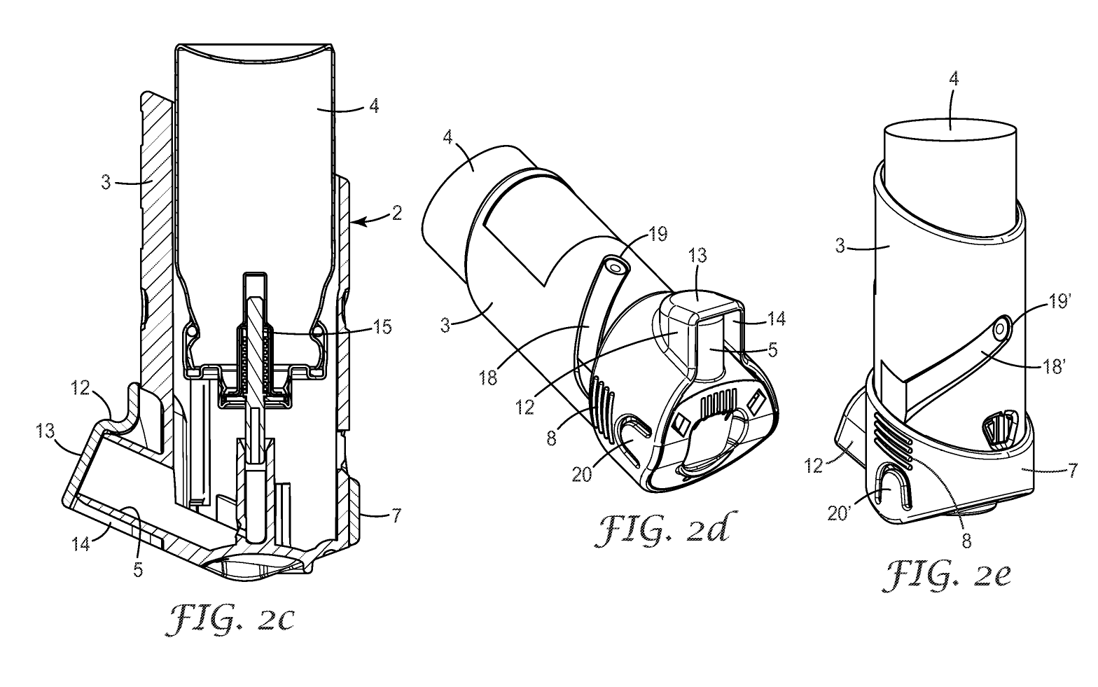

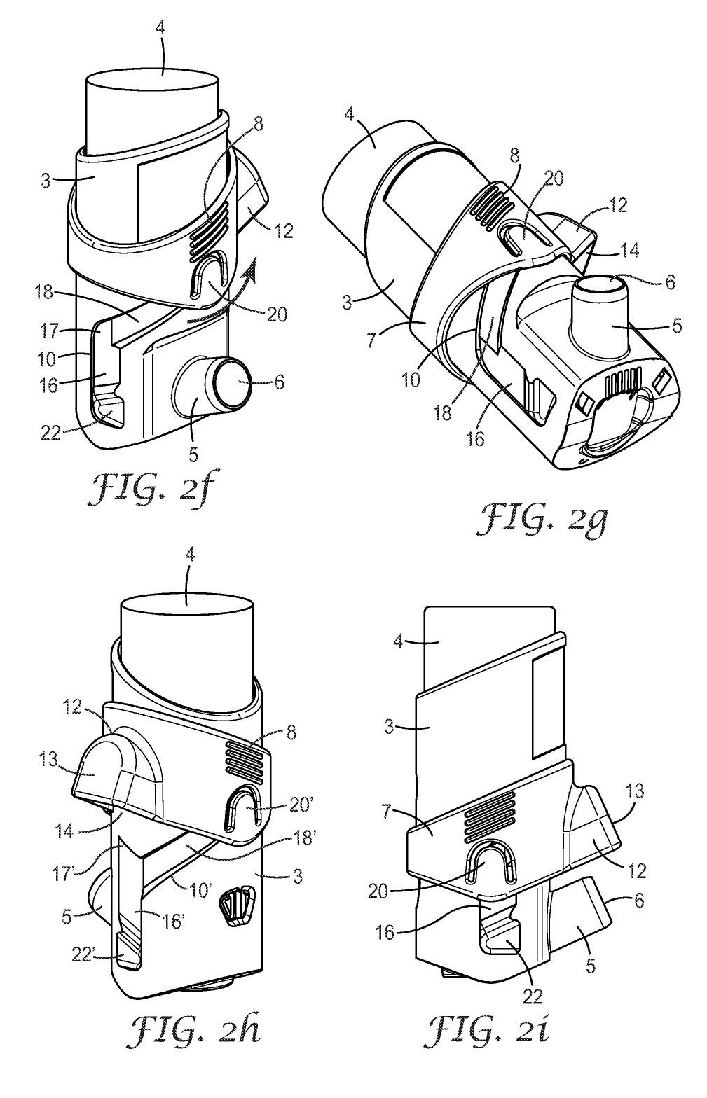

Referring now to FIGS. 2a to 2i, a second exemplary embodiment of an inhaler according to the description is shown. FIGS. 2i and 2j provide illustrations without a cover and canister. The same reference numerals are used for parts corresponding to the previous embodiment.

The inhaler 1 comprises an actuator 2 having a body 3 with a nose piece 5 at a first end of the body and a canister 4 located within the actuator and extending through an opening at a second end of the body. The canister 4 comprises a metering valve generally indicated at 15 (see FIG. 2c) which delivers a metered dose of medicament from the canister to a delivery passage in the nose piece 5. The canister and metering valve are conventional and will not be further described. The actuator 2 further comprises a cover 7.

The inhaler and its actuator with its cover in its closed position can be seen e.g. in FIG. 2a, in an intermediate position e.g. in FIG. 2i, and in its remote location in FIGS. 2f to 2h.

The cover 7 is in the form of a closed ring that encircles and completely surrounds the body 3. The cover 7 is provided with finger grips 8 located on side portions of the cover.

The cover 7 further comprises a forwardly extending projection 12 arranged to extend over the nosepiece 5 in the closed position (see e.g. FIGS. 2a, 2b, and 2d). The projection 12 has an end 13 extending across and occluding the discharge orifice 6 of the nosepiece 5 in the closed position so preventing the ingress of dirt and moisture into the inhaler. The cover 7 substantially encompasses the nose piece 5 where the projection 12 in the closed position of the cover also extends over an upper portion of the nosepiece 5 and over each side of the nosepiece (best seen in FIG. 2d). The cover 7 is a close fit over the nosepiece 5 in the closed position and an aperture 14 is provided in a lower portion of the cover 7 for passage of the nosepiece 5 as the cover 7 is moved to and from the closed position.

The actuator 2 further comprises a pair of opposed profiles 9, 9′ in the form of tracks 10, 10′. (See e.g. FIGS. 2d and 2e as well as FIGS. 2g and 2h showing the front and back side of the actuator with the cover in its closed and opened positions, respectively). The track 10 comprises a groove in the outer surface of the body 3 having a first section 16 on one side of the body that extends longitudinally to a point 17 and a second section 18 that sweeps helically around the outer surface of the body from point 17 to a point 19 spaced above the nosepiece 5 at the front of the body. The track 10′ is similar with a longitudinal first section 16′ that extends to a point 17′ on the opposed side of the body 3 and a helical second section 18′ that extends to a point 19′ at the rear of the body 3.

The cover 7 comprises a pair of tabs 20, 20′ located on opposed sides of the cover 7. Each tab 20, 20′ has an inwardly extending lug 21, 21′ which co-operates with the respective opposed profiles 9, 9′. The tabs 20, 20′ are partially cut out in the cover 7 and are resilient. The lugs 21, 21′ on the inner surface of the cover 7 are received in and slide along the grooves to guide the cover 7 as it moves from the closed position (shown e.g. in FIGS. 2a to 2d) to the remote location (shown in FIGS. 2g to 2h). The engagement of the lugs 21, 21′ in the grooves supports and regulates movement of the cover 7 as the lugs 21, 21′ slide along the grooves and also retains the cover 7 on the body 3. The lugs 21, 21′ are releasably engageable with retainer formations 22, 22′ at one end of the grooves to retain the cover 7 in the closed position and with retainer formations 23, 23′ at the other end of the grooves to retain the cover 7 at the remote location. The tabs 20, 20′ can flex so that the lugs 21, 21′ can engage/disengage the retainer formations resiliently, for example with a snap action, as the cover 7 is moved towards and away from a retained position. In this way, the cover 7 is held in retained positions at the end of travel and the user is provided with a positive indication that the cover 7 is secured.

Starting from the closed position (e.g. see FIG. 2b), the lugs 21, 21′ first travel along the opposed first sections 16, 16′ of the grooves causing the cover 7 to move longitudinally from the closed position to an intermediate position (e.g. see FIG. 2i, and motion indicated by the vertical arrow provided in FIG. 2i). During this movement, the nose piece 5 passes through the aperture 14 in the underside of the projection 12 on the cover 7 so that, in the intermediate position, the projection 12 has cleared the nose piece 5 and is axially spaced from the nosepiece 5.

The lugs 21, 21′ then travel along the helical second sections 18, 18′ of the grooves causing the cover 7 to move helically relative to the body 3 from the intermediate position to the remote location (e.g. see FIG. 2f, and motion indicated by the curved arrow provided in FIG. 2f). During this movement, the cover 7 is displaced axially and angularly relative to the body 3 so that, in the remote location, the projection 12 is axially spaced from and angularly offset relative to the nose piece 5. The cover 7 is thus removed from and angled away from the nosepiece 5 and so does not obstruct the nosepiece or hinder use of the actuator by a patient.

In this embodiment, the projection 12 is angled at substantially 90° to the nosepiece 5 in the cover's remote location. It will be understood that the extent of angular offset may be varied by changing the length and/or inclination of the helical sections 18, 18′ of the grooves.

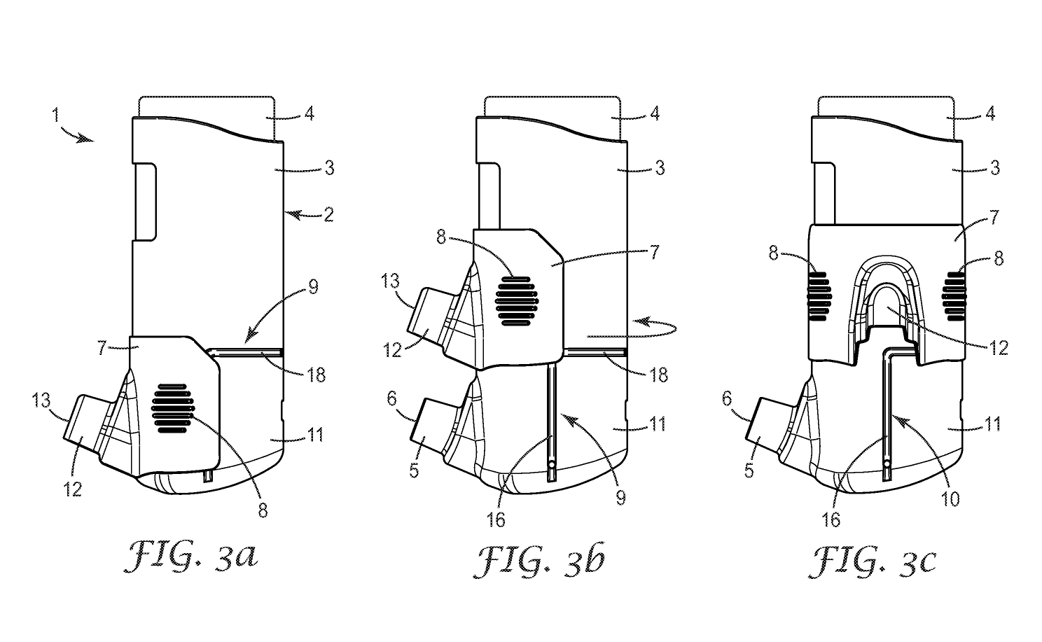

Referring now to FIGS. 3a, 3b and 3c, another exemplary embodiment in accordance with the present description is shown, where the cover is the closed position, an intermediate position and its remote location, respectively. Again like reference numerals are used to indicate parts corresponding to previous embodiments.

In this embodiment the configuration of the grooves forming the profiles 9 (one only visible) on the outer surface of the body 3 is altered so that the second sections 18 of the grooves are transverse to the longitudinal axis of the body 3.

With this arrangement, starting from the closed position (FIG. 3a), the cover 7 initially moves longitudinally relative to the body 3 as the lugs travel along the first sections 16 of the grooves until the cover 7 clears the discharge orifice 6 at the outer end of the nosepiece 5 in the intermediate position (FIG. 3b). The user's pushing/lifting upward force towards the second end of the actuator body is translated into rotational movement where the cover 7 is then rotated about the longitudinal axis of the body through 90 degrees as the lugs travel along the second sections 18 until the cover 7 reaches its remote location (FIG. 3c). In this position the projection 12 on the cover 7 is spaced above and angularly offset relative to the discharge orifice 6 of the nosepiece. The vertical spacing and angular offset can be varied by altering the length of the first and second sections 16, 18 of the grooves. The cover 7 is returned to the closed position by a reverse of the above operation and substantially encompasses the nosepiece 5 in the closed position so preventing ingress of dirt. In other respects, the construction and operation of this embodiment will be understood from previous embodiments.

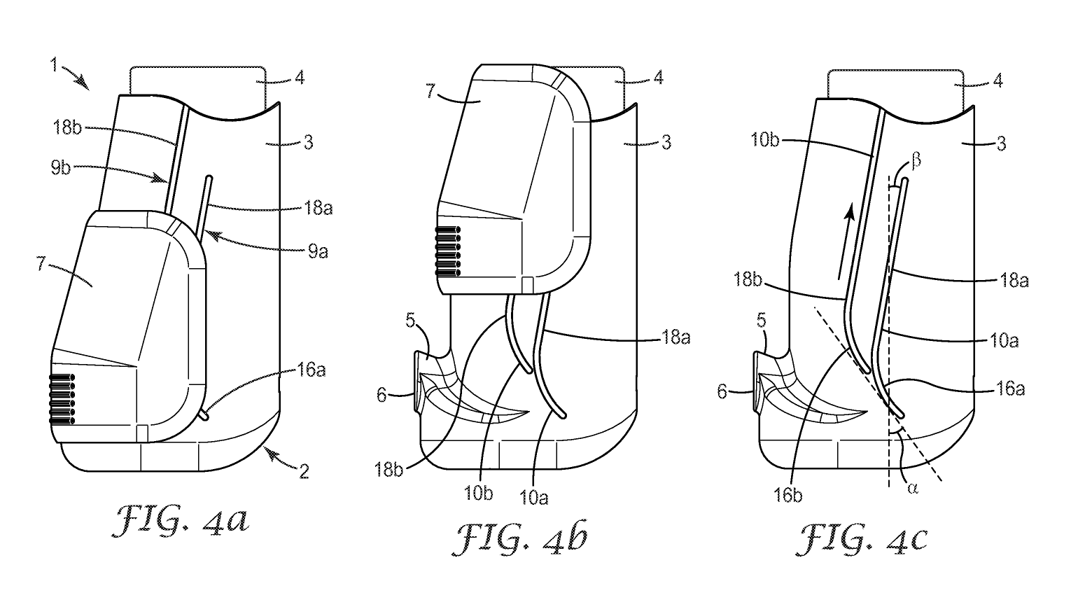

Referring now to FIGS. 4a to 4c, a further exemplary embodiment of an inhaler according to the description is described. The same reference numerals are used for parts corresponding to previous embodiments.

The inhaler 1 comprises an actuator 2 having a body 3 with a nosepiece 5 at the first end of the body and a canister 4 located within the actuator and extending through an opening at the second end of the body. The actuator 2 further comprises a cover 7 which is shown in the closed position in FIG. 4a and in its remote location on the actuator body in FIG. 4b.

The actuator 2 is provided with two pairs of opposed profiles 9a, 9b (only one profile of each pair is visible) in the form of tracks 10a, 10b on opposing sides of the body 3, as can be best seen in FIG. 4c where for illustrative purposes the cover has been left out of the drawing. Each track 10a, 10b comprises a groove in the outer surface of the body 3 and has a first lateral component 16a, 16b that is inclined at an angle α to the longitudinal direction and a second lateral component 18a, 18b that is inclined, in the opposite sense to the first component, at an angle β to the longitudinal direction. In the embodiment shown, the first components 16a, 16b of the tracks are inclined at an angle α to the longitudinal direction about 30° relative to the longitudinal axis of the body in the opposite direction to the inclination of at an angle β about 12° of the second components 18a, 18b. In the illustrated configuration the first lateral movement provides a vertical movement as well as an outward movement transverse to the nose piece. (It will be appreciated that in similar, alternative configurations an angle α of less than 10 degrees would provide little outward movement to traverse the nose piece.) In addition in the illustrated configuration the second lateral movement provides a further vertical movement as well as an inward movement. (It will be appreciated that in similar, alternative configurations that an angle β of less than 5 degrees would bring about little inward movement.) The first components 16a, 16b of the tracks are parallel to each other and the second components 18a, 18b of the tracks are also parallel to each other, and the first and second components are each linked by a smoothly curved intermediate component. The grooves on opposing sides of the body 3 are preferably arranged symmetrically with respect to a vertical plane bisecting the nose piece 5.

The cover 7 is in the form of an open ring such that the sector of the ring subtends an angle of least 180 degrees at the longitudinal axis of the body 3 and partially encircles the body 3 of the actuator. (It will be appreciated that in the present embodiment the extent to which the open ring partially encircles the actuator body depends on the particular position of the cover; here at its remote location the aforesaid angle is around 250 degrees.) The cover 7 is provided with lugs or projections (not shown) on an inner surface that are received in and slide along the grooves to guide the cover 7 as it moves from the closed position shown in FIG. 4a to the remote location on the actuator body shown in FIG. 4b. The engagement of the lugs in the grooves supports and regulates movement of the cover 7 as the lugs slide along the grooves and also retains the cover 7 on the body 3.

The cover 7 is releasably held in the closed position and the remote location by engagement of the lugs with retainer formations at the ends of one or both pairs of grooves. The cover 7 partially encircles the body 3 and can flex so that the lugs can engage/disengage the retainer formations resiliently, for example with a snap action, as the cover 7 is moved towards and away from a retained position. In this way, the cover 7 is held in retained positions at the end of travel and the user is provided with a positive indication that the cover 7 is secured.

Starting from the closed position shown in FIG. 4a, the lugs firstly travel along the grooves corresponding to the opposed first components 16a, 16b causing the cover 7 to move in a diagonal upward direction outwardly of the body 3. After this the lugs negotiate the intermediate linking components of the tracks.

Subsequently, the lugs run along the grooves corresponding to the opposed second lateral components 18a, 18b. During this movement, due to the inclination of the grooves relative to the longitudinal axis of the body 3 draws the cover 7 is drawn towards the body 3 as it moves axially relative to the body 3 so that, in the remote location, the cover 7 is axially spaced from the nose piece 5 which can be used by a patient for the delivery of medication from the canister 4 without the cover 7 interfering with or obstructing use of the inhaler. The cover 7 is returned to the closed position by a reverse of the above operation and completely encompasses the nosepiece 5 in the closed position so preventing ingress of dirt. The provision of two pairs of tracks 10a, 10b on each side of the body 3 of the actuator 2 allows the cover 7 to move through the 2 diagonal directions without tilting, so keeping a fixed orientation relative to the actuator body 3.

In other respects, the construction and operation of this embodiment will be understood from previous embodiments.

It will be understood that the description is not limited to the embodiments above-described and that various modifications can be made without departing from the principles or concepts of the description. For example, in the above described embodiments, the actuator has one or two pairs of profiles for guiding the cover—a single profile may be employed in some circumstances. Alternative arrangements of the profile(s) can be envisaged. Additionally the skilled man will understand that the profile may take a number of forms and that the function of the profile is to guide the cover. The cover may be provided with a projection or lug arranged to run in or along the profile. The skilled man will realise that the profile may comprise a ridge arranged to cooperate with a channel on the cover. Alternatively the cover may be provided with a further profile adapted to cooperate with the profile on the actuator. Whatever the particular profile or profiles or projections are employed, it will be appreciated that they are desirably constructed with materials and dimensions that make for stable retention of the cover on the actuator body and to prevent it from being detached from the actuator during normal patient usage.

Actuators and inhalers according to the description may include appropriately any feature described herein separately or in combination with any other feature(s).

1. An actuator for an inhaler comprising a longitudinally extending body, a delivery passage at (or near to) a first end of the body and a retained cover for the delivery passage, the actuator having at least one profile arranged to guide the cover from a closed position occluding at least a user-opening of the delivery passage to a remote location in which the user-opening of the delivery passage is unoccluded wherein the cover and the at least one profile are configured and arranged such that, in use of the actuator, in the guided movement of the cover from the closed position to the remote location, the cover is glided by the user towards the second end of the body.

2. An actuator according to claim 1, wherein the remote location is on the body of the actuator.

3. An actuator according to claim 1, wherein the cover substantially encompasses the delivery passage in its closed position.

4. An actuator for an inhaler comprising a longitudinally extending body, a delivery passage at (or near to) a first end of the body and a retained cover for the delivery passage, the actuator having at least one profile arranged to guide the cover from a closed position occluding the delivery passage to a remote location on the body, wherein in its closed position the cover substantially encompasses the delivery passage.

5. An actuator according to claim 4, wherein the longitudinally extending body of the actuator defines a longitudinal axis and wherein the cover has a form of a closed ring surrounding the actuator body or a sector of a ring partially surrounding the actuator body.

6. An actuator according to claim 5, wherein the angle subtended by the sector of the ring at the longitudinal axis is greater than 180 degrees, in particular greater than 240 degrees, more particularly greater than 290 degrees, and even more particularly greater than 325 degrees.

7. An actuator according to claim 1 wherein the outer surface of cover comprises one or more user-gripping regions.

8. An actuator according to claim 1 wherein the at least one profile comprises a longitudinal component relative to the actuator body and/or a lateral component relative to the actuator body and/or a helical component relative to the actuator body and/or a rotational component relative to the actuator body.

9. An actuator according to claim 1 wherein the guided movement of the cover from the closed position to the remote location includes a longitudinal component relative to the actuator body and/or a lateral component relative to the actuator body and/or a helical component relative to the actuator body and/or a rotational component relative to the actuator body.

10. An actuator according to claim 1 wherein the guided movement of the cover from the closed position to the remote location does not include a rotational component relative to the actuator body as a first component of said movement.

11. An actuator according to claim 1 wherein the first motion of the cover in its guided movement from the closed position to the remote location is longitudinal or lateral or helical relative to the actuator body.

12. An actuator according to claim 1 wherein the motion of the cover in its guided movement from the closed position to the remote location includes one of the following motions:
longitudinal relative to the actuator body;
longitudinal followed by lateral, helical and/or rotational relative to the actuator body;
lateral relative to the actuator body;
lateral followed by longitudinal, helical, rotational and/or a second lateral motion relative to the actuator body;
helical relative to the actuator body; and
helical following by longitudinal, lateral, rotational and/or a second helical motion relative to the actuator body.

13. An actuator according to claim 1
wherein the at least one profile is provided on an outer surface of the actuator; and/or
wherein the at least one profile is in the form of a track; and/or
wherein the actuator comprises a retainer for holding the cover in the remote location; and/or
wherein the actuator comprises a retainer for holding the cover in the closed position; and/or
wherein the delivery passage of actuator is adapted for oral delivery or adapted for intranasal delivery; and/or
wherein the actuator body is adapted to be able to house and/or support a container containing medicament for delivery to the respiratory system of the user.

14. An actuator according to claim 13 wherein the actuator comprises at least one pair of profiles.

15. An actuator according to claim 14 wherein the at least one pair of profiles are provided on opposing sides of the actuator body.

16. A metered dose inhaler comprising an actuator according to claim 1.

17. A metered dose inhaler according to claim 16, wherein the inhaler is either a pressurized metered dose inhaler or a dry powder inhaler or a nebulizer.
